(19) United States
(12) Patent Application Publication
(10) Pub. No.: US 2010/0240645 A1
(43) Pub. Date: Sep. 23, 2010

(54) COMBINATION OF CHECKPONIT KINASE (CHK) AND TELANGIECTASIA MUTATED (ATM) INHIBITORS FOR THE TREATMENT OF CANCER

(75) Inventors: Graeme Cameron Murray Smith, Cambridgeshire (GB); Sonya Zabludoff, Waltham, MA (US)

Correspondence Address: ASTRAZENECA R&D BOSTON, 35 GATEHOUSE DRIVE, WALTHAM, MA, 02451-1215, US

(73) Assignee: ASTRAZENECA AB, Sodertalje (SE)

(21) Appl. No.: 12/601,332

(22) PCT Filed: May 23, 2008
(86) PCT No.: PCT/GB2008/050373

§ 371 Date: May 17, 2010

Related U.S. Application Data
(60) Provisional application No. 60/940,243, filed on May 25, 2007.

(51) Int. Cl. A61K 31/5377; A61P 35/00; A61P 35/04

(52) U.S. Cl. 514/232.5

(57) ABSTRACT

A combination, comprising a checkpoint kinase (CHK) inhibitor, or a pharmaceutically acceptable salt thereof, and an ataxia telangiectasia mutated (ATM) inhibitor, or a pharmaceutically acceptable salt thereof is described.

### TECHNICAL FIELD

The present invention discloses therapies for treating cancer.

### BACKGROUND OF THE INVENTION

Chemotherapy and radiation exposure are currently the major options for the treatment of cancer, but the therapeutic utility of both these approaches is severely limited by drastic adverse effects on normal tissue, and the frequent development of tumor cell resistance. It is therefore highly desirable to improve the efficacy of cancer treatments in a way that does not increase the toxicity associated with them. In some cases, one way to achieve enhanced efficacy is by employing anticancer agents in combination, wherein said combination causes a better therapeutic effect than that seen with each drug alone.

Combined treatment regimens would add to the therapies available to patients suffering from cancer. For example, in one possible scenario, a drug may act to increase the sensitivity of the malignant cell to the other drug of a combination therapy. In other scenarios, combinations of anticancer agents may have additive, or even synergistic, therapeutic effects.

One particular class of therapeutic agents that is in preclinical studies that has the potential to treat cancer are inhibitors of ATM (ataxia telangiectasia mutated). ATM is a key protein in the detection of DNA double strand breaks (DSBs) and in the signalling of this information to the cell cycle machinery is the kinase ATM (ataxia telangiectasia mutated) (Durocher and Jackson (2001) DNA-PK, ATM and ATR as sensors of DNA damage: variations on a theme? Curr Opin Cell Biol., 13:225-31, Abraham (2001) Cell cycle checkpoint signaling through the ATM and ATR kinases. Genes Dev., 15; 2177-96).

The ATM protein is an ˜350 kDa polypeptide that is a member of the phosphatidylinositol (PI) 3-kinase family of proteins by virtue of a putative kinase domain in its carboxyl-terminal region (Savitsky et al (1995) A single ataxia telangiectasia gene with a product similar to PI-3 kinase. Science, 268:1749-53). Classical PI 3-kinases, such as PI 3-kinase itself, are involved in signal transduction and phosphorylate inositol lipids that act as intracellular second messengers (reviewed in Toker and Cantley (1997) Signalling through the lipid products of phosphoinositide-3-OH kinase, Nature, 387: 673-6).

ATM is the product of the gene mutated in ataxia-telangiectasia (A-T) (Savitsky et al (1995)). A-T is a human autosomal recessive disorder present at an incidence of around 1 in 100,000 in the population. A-T is characterised by a number of debilitating symptoms, including progressive cerebellar degeneration, occulocutaneous telangiectasia, growth retardation, immune deficiencies, cancer predisposition and certain characteristics of premature ageing (Lavin and Shiloh (1997), The genetic defect in ataxia-telangiectasia. Annu. Rev. Immunol., 15:177-202; Shiloh (2001), ATM and ATR: networking cellular responses to DNA damage, Curr. Opin. Genet. Dev., 11:71-7). At the cellular level, A-T is characterised by a high degree of chromosomal instability, radio-resistant DNA synthesis, and hypersensitivity to ionizing radiation (IR) and radiomimetic drugs. In addition, A-T cells are defective in the radiation induced G₁-S, S, and G₂-M cell cycle checkpoints that are thought to arrest the cell cycle in response to DNA damage in order to allow repair of the genome prior to DNA replication or mitosis (Lavin and Shiloh, 1997). This may in part reflect the fact that A-T cells exhibit deficient or severely delayed induction of p53 in response to IR. Indeed, p53-mediated downstream events are also defective in A-T cells following IR exposure. ATM therefore acts upstream of p53 in an IR-induced DNA damage signalling pathway. A-T cells have also been shown to accumulate DNA double-strand breaks (dsbs) after ionizing radiation, suggesting a defect in dsb repair.

It is clear that ATM is a key regulator of the cellular response to DNA DSBs. Therefore the inhibition of this kinase through small molecules will sensitise cells to both ionising radiation and to chemotherapeutics that induce DNA DSBs either directly or indirectly. ATM inhibitors may thus be used as adjuncts in cancer radiotherapy and chemotherapy.

Another particular class of therapeutic agents that has the potential to treat cancer are inhibitors of the checkpoint 1 kinase (CHK1). CHK1 is an important regulatory component of the cell cycle (See, for ex., Prudhomme, Recent Patents on Anti-Cancer Drug Discovery, 2006, 1:55). An individual cell replicates by making an exact copy of its chromosomes, and then segregating these into separate cells. This cycle of DNA replication, chromosome separation and division is regulated by mechanisms within the cell that maintain the order of the steps and ensure that each step is precisely carried out. Key to these processes are the cell cycle checkpoints (Hartwell et al., Science, Nov. 3, 1989, 246(4930):629-34) where cells may arrest to ensure DNA repair mechanisms have time to operate prior to continuing through the cycle into mitosis. Examples of checkpoints that are key in the regulation of the cell cycle are the G1/S checkpoint that is regulated by checkpoint kinase 2 (CHK2) and p53 and the intra-S and G2/M checkpoint that are monitored by the Ser/Thr kinase checkpoint kinase 1 (CHK1). As the cell cycle arrest induced by these checkpoints is a crucial mechanism by which cells can overcome the damage resulting from radio- or chemotherapy, their abrogation by novel agents should increase the sensitivity of tumor cells to DNA damaging therapies. One approach to the design of compounds that abrogate the G2/M checkpoint is to develop inhibitors of the key G2/M regulatory kinase CHK1, and this approach has been shown to work in a number of proof of concept studies. (Koniaras et al., Oncogene, 2001, 20:7453; Luo et al., Neoplasia, 2001, 3:411; Busby et al., Cancer Res., 2000, 60:2108; Jackson et al., Cancer Res., 2000, 60:566).

Several CHK inhibitors have been identified. These compounds include aminopyrazoles, indazoles, tricyclic compounds, ureas, carbamates, diazepinones, pyrimidines, benzimidazole quinolones and macrocyclic compounds. (See, e.g., Prudhomme, Recent Patents on Anti-Cancer Drug Discovery, 2006, 1:55, Janetka et al., Curr Opin Drug Discovery Dev 2007, 10(4)). 2-ureidothiophene compounds and 3-ureidothiophene compounds are described as CHK inhibitors in WO03029241 and WO03028731, respectively. In addition, fused triazolones are described as CHK inhibitors in WO2004/081008. CHK inhibitors also include the thiophene carboxamides disclosed in WO2005/016909; the thiophene carboxamides disclosed in WO 2005/066163; and the substituted heterocycles, described in WO2006/106326.

### SUMMARY OF THE INVENTION

The present invention relates to a combination comprising a checkpoint kinase (CHK) inhibitor, or a pharmaceutically acceptable salt thereof, and an ataxia telangiectasia mutated (ATM) inhibitor, or a pharmaceutically acceptable salt thereof. This combination has been found to be useful for its anti-proliferative (such as anti-cancer) activity and are therefore useful in methods of treatment of the human or animal body.

### BRIEF DESCRIPTION OF THE DRAWINGS

FIG. 1 shows an IC₅₀ plot of a combination of CHK inhibitor and ATM inhibitor with simultaneous addition and exposure in NCI-H460 dominant negative (dn) p53 cell line

FIG. 2 shows an IC₅₀ plot of the combination of a CHK inhibitor followed by ATM inhibitor in an NCI-H460dnp53 cell line.

FIG. 3 shows an IC₅₀ plot of the combination of an ATM inhibitor followed by a CHK inhibitor in an NCI-H460dnp53 cell line.

FIG. 4 shows an IC₅₀ plot of the combination of a CHK inhibitor and an ATM inhibitor with simultaneous addition and exposure in a SW620 cell line.

FIG. 5 shows a combination of CHK inhibitor followed by ATM inhibitor in SW620 cell line.

FIG. 6 shows a combination of an ATM inhibitor followed by a CHK inhibitor in a SW620 cell line.

### DETAILED DESCRIPTION OF THE INVENTION

The present invention relates to a combination comprising a CHK inhibitor, or a pharmaceutically acceptable salt thereof, and an ATM inhibitor, or a pharmaceutically acceptable salt thereof. This combination is useful in methods for the treatment or prophylaxis of cancer. Examples of cancers include oesophageal cancer, myeloma, hepatocellular, pancreatic, cervical cancer, ewings tumour, neuroblastoma, kaposis sarcoma, ovarian cancer, breast cancer, colorectal cancer, prostate cancer, bladder cancer, melanoma, lung cancer-non small cell lung cancer (NSCLC), and small cell lung cancer (SCLC), gastric cancer, head and neck cancer, brain cancer, renal cancer, lymphoma and leukaemia.

CHK Inhibitors

A “CHK inhibitor” refers to any compound or substance that can inhibit the activity of checkpoint 1 kinase and/or the activity of checkpoint 2 kinase. A check inhibitor can be a peptidomimetic, protein, peptide, nucleic acid, small molecule, an antibody or any other agent that can bind and inhibit the activity of CHK. In one example, the check inhibitor is a nucleic acid molecule such as short interfering nucleic acid (siNA), short interfering RNA (siRNA), double-stranded RNA (dsRNA), micro-RNA (miRNA), and short hairpin RNA (shRNA) molecules. Examples of such molecules are disclosed in US 20060216747, the contents of which are incorporated by reference.

In another example, the CHK inhibitor is a small molecule. It is understood that the CHK inhibitors for use in the methods of the present invention include compounds in free form or in the form of a pharmaceutically acceptable salt of the compound or in the form of a pharmaceutically acceptable solvate of the compound or salt. For example, CHK inhibitors include the thiophene carboxamides disclosed in WO2005/066163. These CHK inhibitors can be prepared in a number of ways well known to one skilled in the art of organic synthesis, including, but not limited to, the methods of synthesis described in detail in WO 2005/066163, the entire contents of which are hereby incorporated by reference. Thiophene carboxamides of interest as CHK inhibitors include compounds of the aforementioned WO 2005/066163 as shown in Formula (I):

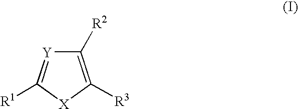

wherein:
X is selected from NH, S and O;
Y is selected from CH or N;
R¹ is selected from cyano, isocyano, C₁₋₆alkyl, —NR¹¹R¹², C₁₋₆alkoxy, C₂₋₆alkenyl, C₂₋₆alkynyl, cycloalkyl, cycloalkenyl, aryl, and heterocyclyl, provided R¹ is not thienyl; and wherein R¹ may be optionally substituted on one or more carbon atoms by one or more R⁹; and wherein if said R¹ contains an —NH— moiety, the nitrogen of said moiety may be optionally substituted by a group selected from R¹⁰;
R² and R³ are each independently selected from —C(═O)NR⁶R⁷, —SO₂NR¹⁶R¹⁷, —NHC(═O)NHR⁴, and —NHC(═NR⁸)NH₂;
R⁴ is selected from H, OH, —NR¹¹R¹², benzyl, C₁₋₆alkoxy, cycloalkyl, cycloalkenyl, aryl, heterocyclyl, mercapto, CHO, —COaryl, —CO(C₁₋₆alkyl) —CONR³⁰R³¹, —CO₂(C₁₋₆alkyl), —CO₂aryl, —CO₂NR³⁰R³¹, —Salkyl, —SO(C₁₋₆alkyl), —SO₂(C₁₋₆alkyl), —Saryl, —SOaryl, —SO₂aryl, —SO₂NR³⁰R³¹, and —(C₁₋₆alkyl)SO₂ NR³⁰R³¹ wherein R⁴ may be optionally substituted on one or more carbon atoms by one or more R¹⁵; and wherein if said heterocyclyl contains a —NH— moiety, the nitrogen may be optionally substituted by a group selected from R¹⁴;
R⁶ and R⁷ are each independently selected from H, OH, OCH₃, C₁₋₆alkoxy, —NH₂, —NHCH₃, —N(CH₃)₂, (C₁₋₃alkyl)NR¹¹R¹², —CH₂CH₂OH, cycloalkyl, and a 5, 6, or 7-membered heterocyclyl ring containing at least one nitrogen atom, provided R⁶ and R⁷ are not both H; alternatively R⁶ and R⁷ taken together with the N to which they are attached form a heterocyclic ring; wherein R⁶ and R⁷ independently of each other may be optionally substituted on one or more carbon atoms by one or more R¹⁸; and wherein if said heterocyclyl contains a —NH— moiety, the nitrogen of said moiety may be optionally substituted by a group selected from R¹⁹;
R⁸ is selected from cyano, isocyano, —SO₂(C₁₋₆alkyl), —SO₂-aryl; —SO₂cycloalkyl, —SO₂cycloalkenyl, —SO₂heterocyclyl, and CF₃; wherein R⁸ may be optionally substituted on one or more carbon atoms by one or more R²³;
R⁹, R¹⁵, R¹⁸, R²³, R²⁴ and R³³ are each independently selected from halogen, nitro, —NR³⁰R³¹, cyano, isocyano, C₁₋₆alkyl, C₂₋₆alkenyl, C₂₋₆alkynyl, aryl, cycloalkyl, heterocyclyl, hydroxy, keto(═O), —O(C₁₋₆alkyl), —Oaryl, —OCOalkyl, —NHCHO, —N(C₁₋₆alkyl)CHO, —NHCONR³⁰R³¹, —N(C₁₋₆alkyl)CONR³⁰R³¹, —NHCOalkyl, —NHCO₂(C₁₋₆alkyl); —NHCO₂H, —N(C₁₋₆alkyl)CO(C₁₋₆alkyl), —NHSO₂(C₁₋₆alkyl), carboxy, -amidino, —CHO, —CONR³⁰R³¹, —CO(C₁₋₆alkyl), —COheterocyclyl, —COcycloalkyl, —CO₂H, —CO₂(C₁₋₆alkyl), —CO₂(aryl), —CO₂(NR³⁰R³¹), mercapto, —S(C₁₋₆alkyl), —SO(C₁₋₆alkyl), —SO₂(C₁₋₆alkyl), —SO₂NR³⁰R³¹; wherein R⁹, R¹⁵, R¹⁸, R²³, R²⁴ and R³³ independently of each other may be optionally substituted on carbon by one or more R²⁰ and on nitrogen of any moiety that contains an NH or NH₂ by R²¹;
R¹⁰, R¹⁴, R¹⁹, R²⁵ and R³⁴ are each independently selected from halogen, nitro, —NR³⁰R³¹, cyano, isocyano, C₁₋₆alkyl, C₂₋₆alkenyl, C₂₋₆alkynyl, aryl, cycloalkyl, heterocyclyl, hydroxy, keto(═O), —O(C₁₋₆alkyl), —Oaryl, —OCOalkyl, —NHCHO, —N(C₁₋₆alkyl)CHO, —NHCONR³⁰R³¹, —N(C₁₋₆alkyl)CONR³⁰R³¹, —NHCOalkyl, —NHCO₂(C₁₋₆alkyl); —NHCO₂H, —N(C₁₋₆alkyl)CO(C₁₋₆alkyl), —NHSO₂(C₁₋₆alkyl), carboxy, -amidino, —CHO, —CONR³⁰R³¹, —CO(C₁₋₆alkyl), —COheterocyclyl, —COcycloalkyl, —CO₂H, —CO₂(C₁₋₆alkyl), —CO₂(aryl), —CO₂ mercapto, —S(C₁₋₆alkyl), —SO(C₁₋₆alkyl), —SO₂(C₁₋₆alkyl), —SO₂NR³⁰R³¹; wherein R¹⁰, R¹⁴, R¹⁹, R²⁵ and R³⁴ independently of each other may be optionally substituted on carbon by one or more R²² and on nitrogen of any moiety that contains an NH or NH₂ by R²³;
R¹¹ and R¹² are independently selected from H, C₁₋₆alkyl, cycloalkyl, aryl, heterocyclyl; alternatively R¹¹ and R¹² taken together with the N to which they are attached form a heterocyclic ring; wherein R¹¹ and R¹² independently of each other may be optionally substituted on carbon by one or more R³³, and wherein if said heterocyclyl contains a —NH— moiety, the nitrogen of said moiety may be optionally substituted by a group selected from R³⁴;
R¹⁶ and R¹⁷ are each independently selected from H, OH, OCH₃, C₁₋₆alkoxy, NH₂, —NHCH₃, —N(CH₃)₂, (C₁₋₃alkyl)NR¹¹R¹², —CH₂CH₂OH, cycloalkyl, aryl, or a 5, 6 or 7-membered heterocyclyl ring containing at least one nitrogen atom, provided R¹⁶ and R¹⁷ are not both H; alternatively R¹⁶ and R¹⁷ taken together with the N to which they are attached form an optionally substituted heterocyclic ring; wherein R¹⁶ and R¹⁷ independently of each other may be optionally substituted on one or more carbon atoms by one or more R²⁴; and wherein if said heterocyclyl contains an —NH— moiety, the nitrogen of said moiety may be optionally substituted by a group selected from R²⁵;
R²⁰, R²² and R³² are each independently selected from halogen, nitro, —NR³⁰R³¹, cyano, isocyano, C₁₋₆alkyl, C₂₋₆alkenyl, C₂₋₆alkynyl, aryl, cycloalkyl, heterocyclyl, hydroxy, keto(═O), —O(C₁₋₆alkyl), —Oaryl, —OCOalkyl, —NHCHO, —N(C₁₋₆alkyl)CHO, —NHCONR³⁰R³¹, —N(C₁₋₆alkyl)CONR³⁰R³¹, —NHCOalkyl, —NHCO₂(C₁₋₆alkyl); —NHCO₂H, —N(C₁₋₆alkyl)CO(C₁₋₆alkyl), —NHSO₂(C₁₋₆alkyl), carboxy, -amidino, —CHO, —CONR³⁰R³¹, —CO(C₁₋₆alkyl), —COheterocyclyl, —COcycloalkyl, —CO₂H, —CO₂(C₁₋₆alkyl), —CO₂(aryl), —CO₂(NR³⁰R³¹), mercapto, —S(C₁₋₆alkyl), —SO(C₁₋₆alkyl), —SO₂(C₁₋₆alkyl), —SO₂NR³⁰R³¹; wherein R²⁰, R²¹ and R³² independently of each other may be optionally substituted on carbon by one or more R²⁶ and on nitrogen of any moiety that contains an NH or NH₂ by R²⁷;
R²¹, R²³ and R³⁵ are each independently selected from halogen, nitro, —NR³⁰R³¹, cyano, isocyano, C₁₋₆alkyl, C₂₋₆alkenyl, C₂₋₆alkynyl, aryl, cycloalkyl, heterocyclyl, hydroxy, keto(═O), —O(C₁₋₆alkyl), —Oaryl, —OCOalkyl, —NHCHO, —N(C₁₋₆alkyl)CHO, —NHCONR³⁰R³¹, —N(C₁₋₆alkyl)CONR³⁰R³¹, —NHCOalkyl, —NHCO₂(C₁₋₆alkyl); —NHCO₂H, —N(C₁₋₆alkyl)CO(C₁₋₆alkyl), —NHSO₂(C₁₋₆alkyl), carboxy, -amidino, —CHO, —CONR³⁰R³¹, —CO(C₁₋₆ alkyl), —COheterocyclyl, —COcycloalkyl, —CO₂H —CO₂(C₁₋₆ alkyl), —CO₂(aryl), —CO₂ (NR³⁰R³¹), mercapto, —S(C₁₋₆alkyl), —SO(C₁₋₆alkyl), —SO₂ (C₁₋₆alkyl), —SO₂NR³⁰R³¹; wherein R²¹, R²³ and R³⁵ independently of each other may be optionally substituted on carbon by one or more R²⁸ and on nitrogen of any moiety that contains an NH by R²⁹;
R²⁶ and R²⁸ are each independently selected from halogen, nitro, —NR³⁰R³¹, cyano, isocyano, C₁₋₆alkyl, C₂₋₆alkenyl, C₂₋₆alkynyl, aryl, cycloalkyl, heterocyclyl, hydroxy, keto(═O), —O(C₁₋₆alkyl), —Oaryl, —OCOalkyl, —NHCHO, —N(C₁₋₆alkyl)CHO, —NHCONR³⁰R³¹, —N(C₁₋₆alkyl)CONR³⁰R³¹, —NHCOalkyl, —NHCO₂(C₁₋₆alkyl); —NHCO₂H, —N(C₁₋₆alkyl)CO(C₁₋₆alkyl), —NHSO₂(C₁₋₆alkyl), carboxy, -amidino, —CHO, —CONR³⁰R³¹, —CO(C₁₋₆alkyl), —COheterocyclyl, —COcycloalkyl, —CO₂H, —CO₂(C₁₋₆alkyl), —CO₂(aryl), —CO₂(NR³⁰R³¹), mercapto, —S(C₁₋₆alkyl), —SO(C₁₋₆alkyl), —SO₂(C₁₋₆alkyl), —SO₂NR³⁰R³¹;
R²⁷ and R²⁹ are each independently selected from halogen, nitro, —NR³⁰R³¹, cyano, isocyano, C₁₋₆alkyl, C₂₋₆alkenyl, C₂₋₆alkynyl, aryl, cycloalkyl, heterocyclyl, hydroxy, keto(═O), —O(C₁₋₆alkyl), —Oaryl, —OCOalkyl, —NHCHO, —N(C₁₋₆alkyl)CHO, —NHCONR³⁰R³¹, —N(C₁₋₆alkyl)CONR³⁰R³¹, —NHCOalkyl, —NHCO₂(C₁₋₆alkyl); —NHCO₂H, —N(C₁₋₆alkyl)CO(C₁₋₆alkyl), —NHSO₂(C₁₋₆alkyl), carboxy, -amidino, —CHO, —CONR³⁰R³¹, —CO(C₁₋₆alkyl), —COheterocyclyl, —COcycloalkyl, —CO₂H, —CO₂(C₁₋₆alkyl), —CO₂(aryl), —CO₂(NR³⁰R³¹), mercapto, —S(C₁₋₆alkyl), —SO(C₁₋₆alkyl), —SO₂(C₁₋₆alkyl), —SO₂NR³⁰R³¹;
R³⁰ and R³¹ are each independently selected from halogen, nitro, —NH₂, cyano, isocyano, C₁₋₆alkyl, C₂₋₆alkenyl, C₂₋₆alkynyl, aryl, cycloalkyl, heterocyclyl, hydroxy, keto(═O), —O(C₁₋₆alkyl), —Oaryl, —OCOalkyl, —NHCHO, —N(C₁₋₆alkyl)CHO, —NHCONR¹¹R¹², —N(C₁₋₆alkyl)CONR¹¹R¹², —NHCOalkyl, —NHCO₂(C₁₋₆alkyl); —NHCO₂H, —N(C₁₋₆alkyl)CO(C₁₋₆alkyl), —NHSO₂(C₁₋₆alkyl), carboxy, -amidino, —CHO, —CONR³⁰R³¹, —CO(C₁₋₆alkyl), —COheterocyclyl, —COcycloalkyl, —CO₂H, —CO₂(C₁₋₆alkyl), —CO₂(aryl), —CO₂(NR³⁰R³¹), mercapto, —S(C₁₋₆alkyl), —SO(C₁₋₆alkyl), —SO₂(C₁₋₆alkyl), —SO₂NR¹¹R¹²; wherein R³⁰ and R³¹ independently of each other may be optionally substituted on carbon by one or more R³²; and wherein if said heterocyclyl contains a —NH— or NH₂ moiety, the nitrogen of said moiety may be optionally substituted by a group selected from R³⁵;
or a pharmaceutically acceptable salt thereof;
provided that when X is S; Y is CH; R₂ is C(═O)NR⁶R⁷; and R³ is NHC(═O)NHR⁴; then R¹ cannot be

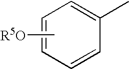

wherein R⁵ is selected from H, optionally substituted carbocyclyl, or optionally substituted C₁₋₆alkyl; with the further proviso that said compound is not
5-Methyl-2-ureido-thiophene-3-carboxylic acid (1-ethyl-piperidin-3-yl)-amide;
[3-((S)-3-Amino-azepane-1-carbonyl)-5-ethyl-thiophen-2-yl]-urea;
2-Morpholin-4-yl-4-ureido-thiazole-5-carboxylic acid (S)-piperidin-3-ylamide;
2-Methyl-5-ureido-oxazole-4-carboxylic acid (S)-piperidin-3-ylamide;
5-(4-Chloro-phenyl)-3-{3-[(R)-1-(2,2,2-trifluoro-acetyl)-piperidin-3-yl]-ureido}-thiophene-2-carboxylic acid (S)-piperidin-3-ylamide; or
N-(3-{[(3S)-3-aminoazepan-1-yl]carbonyl}-5-pyridin-2-yl-2-thienyl)urea.
   Compounds of Formula (I) which are of particular interest include the following:
5-(3-Fluoro-phenyl)-3-ureido-thiophene-2-carboxylic acid (S)-piperidin-3-ylamide;
5-Phenyl-2-ureido-thiophene-3-carb oxylic acid (S)-piperidin-3-ylamide;
5-(3,5-Difluoro-phenyl)-2-ureido-thiophene-3-carb oxylic acid (S)-piperidin-3-ylamide;
5-(4-Fluoro-phenyl)-2-ureido-thiophene-3-carb oxylic acid (S)-piperidin-3-ylamide;
5-(4-Chloro-phenyl)-2-ureido-thiophene-3-carb oxylic acid (S)-piperidin-3-ylamide;
5-(3-Chloro-phenyl)-2-ureido-thiophene-3-carb oxylic acid (S)-piperidin-3-ylamide;
5-[4-(Piperidine-1-carbonyl)-phenyl]-2-ureido-thiophene-3-carb oxylic acid (S)-piperidin-3-ylamide;
5-(4-Cyano-phenyl)-3-ureido-thiophene-2-carb oxylic acid (S)-piperidin-3-ylamide;
5-[4-(Piperidine-1-carbonyl)-phenyl]-3-ureido-thiophene-2-carb oxylic acid (S)-piperidin-3-ylamide;
5-(3,4-Difluoro-phenyl)-3-ureido-thiophene-2-carb oxylic acid (S)-piperidin-3-ylamide;
5-(3-Chloro-phenyl)-3-ureido-thiophene-2-carb oxylic acid (S)-piperidin-3-ylamide;
5-(2,3-Difluoro-phenyl)-3-ureido-thiophene-2-carb oxylic acid (S)-piperidin-3-ylamide;
5-(2,4-Difluoro-phenyl)-3-ureido-thiophene-2-carb oxylic acid (S)-piperidin-3-ylamide;
5-(3,5-Difluoro-phenyl)-3-ureido-thiophene-2-carb oxylic acid (S)-piperidin-3-ylamide;
5-Phenyl-3-ureido-thiophene-2-carb oxylic acid (S)-piperidin-3-ylamide;
5-(4-Chloro-phenyl)-3-ureido-thiophene-2-carb oxylic acid (S)-piperidin-3-ylamide.
   Additional CHK inhibitors include the substituted heterocycles disclosed in WO2006/106326, which is incorporated by reference herein.

Unless specified otherwise within this specification, the nomenclature used in this specification generally follows the examples and rules stated in Nomenclature of Organic Chemistry, Sections A, B, C, D, E, F, and H, Pergamon Press, Oxford, 1979, which is incorporated by references herein for its exemplary chemical structure names and rules on naming chemical structures.

Definitions for use with CHK inhibitors of Formula (I) above. As used in this application, the term “optionally substituted,” means that substitution is optional and therefore it is possible for the designated atom to be unsubstituted. In the event a substitution is desired then such substitution means that any number of hydrogens on the designated atom is replaced with a selection from the indicated group, provided that the normal valency of the designated atom is not exceeded, and that the substitution results in a stable compound. For example when a substituent is keto (i.e., ═O), then 2 hydrogens on the atom are replaced. When a group is indicated to be “optionally substituted” or “substituted” unless otherwise expressly stated examples of suitable substituents include the following:

halogen, nitro, amino, cyano, trifluoromethyl, methyl, ethyl, alkyl, alkenyl, alkynyl, haloalkyl, alkoxy, hydroxy, alkylhydroxy, carbonyl, keto, —CH(OH)CH₃, —CH₂NH-alkyl-OH, alkyl-(OH)CH₃, —Oalkyl, —OCOalkyl, —NHCHO, —N-(alkyl)-CHO, —NH—CO-amino, —N-(alkyl)—CO-amino, —NH—COalkyl, —N-(alkyl)-COalkyl, -carboxy, -amidino, —CO-amino, —CO-alkyl, —CO₂alkyl, mercapto, -Salkyl, —SO(alkyl), —SO₂(alkyl), —SO₂-amino, -alkylsulfonylamino, phenyl, cycloalkyl, heterocyclic and heteroaryl, -alkly-NH-cycloalkyl, -alkyl-NH-optionally substituted heterocyclyl, -alkyl-NH-alkyl-OH, —C(═O)OC(CH₃)₃, —N(CH₃)₂, -alkyl-NH-alkyl-optionally substituted heterocyclyl, alkyl-aryl, alkyl-polycyclyl, alkyl-amino, alkyl-hydroxy, —CH₂NH-alkyl-heterocyclyl, —CH₂NHCH₂CH(CH₃)₂ If the group to be substituted is a ring, the optional substituents could also be selected from: vicinal —O(alkyl)O—, vicinal —OC(haloalkyl)O—, vicinal —CH₂O(alkyl)O—, vicinal —S(alkyl)S— and —O(alkyl)S—. Each of these substituents can, themselves, be further substituted. Suitable examples of such further substitution include any of the foregoing suitable substituents.

The term “hydrocarbon” used alone or as a suffix or prefix, refers to any structure comprising only carbon and hydrogen atoms up to 14 carbon atoms.

The term “hydrocarbon radical” or “hydrocarbyl” used alone or as a suffix or prefix, refers to any structure as a result of removing one or more hydrogens from a hydrocarbon.

The term “alkyl” used alone or as a suffix or prefix, refers to monovalent straight or branched chain hydrocarbon radicals comprising 1 to about 12 carbon atoms. Unless otherwise specified, “alkyl” general includes both saturated alkyl and unsaturated alkyl.

The term “alkenyl” used alone or as suffix or prefix, refers to a monovalent straight or branched chain hydrocarbon radical having at least one carbon-carbon double bond and comprising at least 2 up to about 12 carbon atoms.

The term “alkylene” used alone or as suffix or prefix, refers to divalent straight or branched chain hydrocarbon radicals comprising 1 to about 12 carbon atoms, which serves to links two structures together.

The term “alkynyl” used alone or as suffix or prefix, refers to a monovalent straight or branched chain hydrocarbon radical having at least one carbon-carbon triple bond and comprising at least 2 up to about 12 carbon atoms.

The term “cycloalkyl,” used alone or as suffix or prefix, refers to a monovalent ring-containing hydrocarbon radical comprising at least 3 up to about 12 carbon atoms. When cycloalkyl contains more than one ring, the rings may be fused or unfused and include bicyclo radicals. Fused rings generally refer to at least two rings sharing two atoms therebetween.

The term “cycloalkenyl” used alone or as suffix or prefix, refers to a monovalent ring-containing hydrocarbon radical having at least one carbon-carbon double bond and comprising at least 3 up to about 12 carbon atoms. When cycloalkenyl contains more than one ring, the rings may be fused or unfused and include bicyclo radicals.

The term “aryl” used alone or as suffix or prefix, refers to a hydrocarbon radical having one or more polyunsaturated carbon rings having aromatic character, (e.g., 4n+2 delocalized electrons) and comprising 5 up to about 14 carbon atoms, wherein the radical is located on a carbon of the aromatic ring.

The term “alkoxy” used alone or as a suffix or prefix, refers to radicals of the general formula —O—R, wherein —R is selected from a hydrocarbon radical. Exemplary alkoxy includes methoxy, ethoxy, propoxy, isopropoxy, butoxy, t-butoxy, isobutoxy, cyclopropylmethoxy, allyloxy, and propargyloxy.

The term “carbocyclyl” is intended to include both alicyclic and aromatic ring structures wherein the closed ring is made of carbon atoms. These may include fused or bridged polycyclic systems. Carbocyclyls may have from 3 to 10 carbon atoms in their ring structure, and often have 3, 4, 5, 6 and 7 carbon atoms in the ring structure. For example, “C₃₋₇ carbocyclyl” denotes such groups as cyclopropyl, cyclobutyl, cyclopentyl, cyclohexyl, cycloheptyl, cyclopentadiene or phenyl.

The term” or “heterocyclyl” used alone or as a suffix or prefix, refers to a ring-containing structure or molecule having one or more multivalent heteroatoms, independently 14 atoms in the ring(s). Heterocyclyl may be saturated or unsaturated, containing one or more double bonds, and heterocyclyl may contain more than one ring. When a heterocyclyl contains more than one ring, the rings may be fused or unfused. Fused rings generally refer to at least two rings sharing two atoms therebetween. Heterocyclyl may have aromatic character or may not have aromatic character.

Examples of heterocyclyls include, but are not limited to, 1H-indazolyl, 2-pyrrolidonyl, 2H, 6H-1,5,2-dithiazinyl, 2H-pyrrolyl, 3H-indolyl, 4-piperidonyl, 4aH-carbazolyl, 4H-quinolizinyl, 6H-1,2,5-thiadiazinyl, acridinyl, azepanyl, azetidinyl, aziridinyl, azocinyl, benzimidazolyl, benzofuranyl, benzofuranyl, benzothiofuranyl, benzothiophenyl, benzodioxolyl, benzoxazolyl, benzthiophenyl, benzthiazolyl, benzotriazolyl, benzotetrazolyl, benzisoxazolyl, benzthiazole, benzisothiazolyl, benzimidazolyls, benzimidazalonyl, carbazolyl, 4aH-carbazolyl, b-carbolinyl, chromanyl, chromenyl, cinnolinyl, decahydroquinolinyl, 2H,6H-1,5,2-dithiazinyl, dioxolanyl, furyl, 2,3-dihydrofuranyl, 2,5-dihydrofuranyl, dihydrofuro[2,3-b]tetrahydrofuranyl, furanyl, furazanyl, homopiperidinyl, imidazolyl, imidazolidinyl, imidazolidinyl, imidazolinyl, imidazolyl, 1H-indazolyl, indolenyl, indolinyl, indolizinyl, indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxiranyl, oxazolidinylperimidinyl, phenanthridinyl, phenanthrolinyl, phenarsazinyl, phenazinyl, phenothiazinyl, phenoxathiinyl, phenoxazinyl, phthalazinyl, piperazinyl, piperidinyl, piperidinyl, pteridinyl, piperidonyl, 4-piperidonyl, purinyl, pyranyl, pyrrolidinyl, pyrrolinyl, pyrrolidinyl, pyrazinyl, pyrazolidinyl, pyrazolinyl, pyrazolyl, pyridazinyl, pyridooxazolyl, pyridoimidazolyl, pyridothiazolyl, pyridinyl, N-oxide-pyridinyl, pyridyl, pyrimidinyl, pyrrolidinyl, pyrrolinyl, pyrrolyl, pyridinyl, quinazolinyl, quinolinyl, 4H-quinolizinyl, quinoxalinyl, quinuclidinyl, carbolinyl, tetrahydrofuranyl, tetrahydroisoquinolinyl, thiophanyl, thiotetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, thiiranyl, triazinyl, 1,2,3-triazolyl, 1,2,4-triazolyl, 1,2,5-triazolyl, 1,3,4-triazolyl, and xanthenyl.

The terms “seven-membered”, “six-membered” and “five-membered” used as prefix refers to groups having a ring that contains, respectively, seven, six, and five ring atoms.

The term “substituted” used as a suffix of a first structure, molecule or group, followed by one or more names of chemical groups refers to a second structure, molecule or group, which is a result of replacing one or more hydrogens of the first structure, molecule or group with the one or more named chemical groups. For example, a “phenyl substituted by nitro” refers to nitrophenyl.

The term “amine” or “amino” used alone or as a suffix or prefix, refers to radicals of the general formula —NRR′, wherein R and R′ are independently selected from hydrogen or a hydrocarbon radical.

The term halogen includes fluorine, chlorine, bromine and iodine.

“Halogenated,” used as a prefix of a group, means one or more hydrogens on the group are replaced with one or more halogens.

“RT” or “rt” means room temperature.

When any variable (e.g., R¹, R⁴, R^{a}, R^{e} etc.) occurs more than one time in any constituent or formula for a compound, its definition at each occurrence is independent of its definition at every other occurrence. Thus, for example, if a group is shown to be substituted with 0-3 R¹, then said group may optionally be substituted with 0, 1, 2 or 3 R¹ groups and R^{e} at each occurrence is selected independently from the definition of R^{e}. Also, combinations of substituents and/or variables are permissible only if such combinations result in stable compounds.

A variety of compounds in the present invention may exist in particular geometric or stereoisomeric forms. The present invention takes into account all such compounds, including cis- and trans isomers, R- and S-enantiomers, diastereomers, (D)-isomers, (L)-isomers, the racemic mixtures thereof, and other mixtures thereof, as being covered within the scope of this invention. Additional asymmetric carbon atoms may be present in a substituent such as an alkyl group. All such isomers, as well as mixtures thereof, are intended to be included in this invention. The compounds herein described may have asymmetric centers. Compounds of the present invention containing an asymmetrically substituted atom may be isolated in optically active or racemic forms. It is well known in the art how to prepare optically active forms, such as by resolution of racemic forms or by synthesis from optically active starting materials. When required, separation of the racemic material can be achieved by methods known in the art. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. All chiral, diastereomeric, racemic forms and all geometric isomeric forms of a structure are intended, unless the specific stereochemistry or isomeric form is specifically indicated.

When a bond to a substituent is shown to cross a bond connecting two atoms in a ring, then such substituent may be bonded to any atom on the ring. When a substituent is listed without indicating the atom via which such substituent is bonded to the rest of the compound of a given formula, then such substituent may be bonded via any atom in such substituent. Combinations of substituents and/or variables are permissible only if such combinations result in stable compounds.

When a circle is shown within a ring structure, i.e.

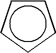

it indicates that the ring system is aryl or heteroaryl.

As used herein, the phrase “protecting group” means temporary substituents which protect a potentially reactive functional group from undesired chemical transformations. Examples of such protecting groups include esters of carboxylic acids, silyl ethers of alcohols, and acetals and ketals of aldehydes and ketones respectively. The field of protecting group chemistry has been reviewed (Greene, T. W.; Wuts, P. G. M. Protective Groups in Organic Synthesis, 3^{rd} ed.; Wiley: New York, 1999).

As used herein, “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, maleic, tartaric, citric, ascorbic, palmitic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, the disclosure of which is hereby incorporated by reference.

Methods of making these compounds are known in the art as described in WO2005/066163, which is incorporated herein by reference.

Atm Inhibitors:

An “ATM inhibitor” refers to any agent that can inhibit the activity of ATM. An ATM inhibitor can be a peptidomimetic, protein, peptide, nucleic acid, small molecule, an antibody or any other agent that can bind and inhibit the activity of ATM. In one example, the ATM inhibitor is an antibody. In another example, the ATM inhibitor is a small molecule. For example, ATM small molecule inhibitors have been previously described such as described in published PCT application, WO 03/070726 (incorporated herein by reference). The ATM inhibitors have the following general structure:

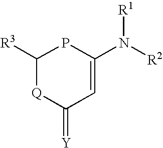

Further ATM inhibitors from within that broad class of compounds are described in the published PCT application, WO 2005/016919 (incorporated herein by reference). ATM inhibitors described in WO 03/070726 and WO 2005/016919 were shown to sensitise cells to ionising radiation or DNA double strand break chemotherapies and to inhibit retroviral transduction and replication.

In one aspect of the invention, the ATM inhibitor can be selected from a compound of Formula (Ib):

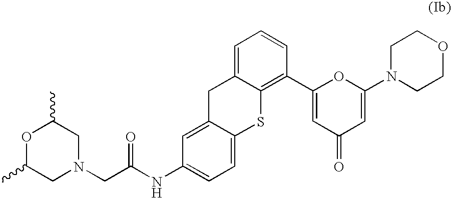

and isomers, salts, solvates, chemically protected forms and prodrugs thereof. The compound of formula (I) has the chemical name: 2-(2,6-Dimethyl-morpholin-4-yl)-N-[5-(6-morpholin-4-yl-4-oxo-4H-pyran-2-yl)-9H-thioxanthen-2-yl]-acetamide.
In particular, the following isomers of the compound of formula (Ib) are of interest:

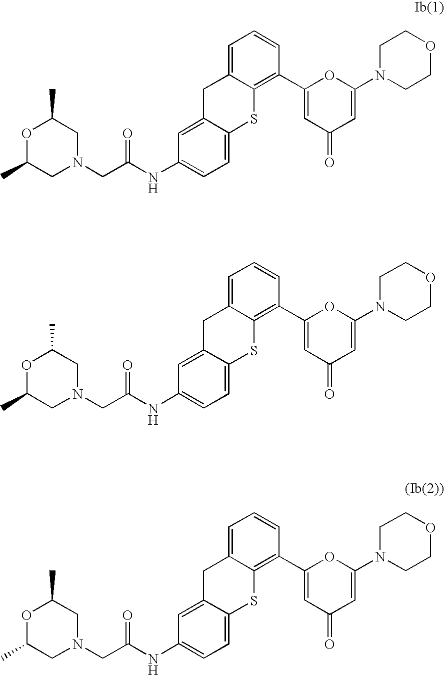

The compound of formula (Ib(1)) is the cis-form, 2-((2R,6S)-2,6-dimethyl-morpholin-4-yl)-N-[5-(6-morpholin-4-yl-4-oxo-4H-pyran-2-yl)-9H-thioxanthen-2-yl]-acetamide. The two diastereoisomers of the compound of formula (Ib(2)) are the trans-form, 2-((2S,6S)-2,6-dimethyl-morpholin-4-yl)-N-[5-(6-morpholin-4-yl-4-oxo-4H-pyran-2-yl)-9H-thioxanthen-2-yl]-acetamide and 2-((2R,6R)-2,6-dimethyl-morpholin-4-yl)-N-[5-(6-morpholin-4-yl-4-oxo-4H-pyran-2-yl)-9H-thioxanthen-2-yl]-acetamide.

Methods of making these compounds are known in the art, for example, as described in PCT/GB2006/003230, which is incorporated herein by reference.

Definitions for use with ATM inhibitors of formula (Ib) above:

Isomers, Salts, Solvates, Protected Forms, and Prodrugs

Certain compounds may exist in one or more particular geometric, optical, enantiomeric, diasteriomeric, epimeric, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and l-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

Note that, except as discussed below for tautomeric forms, specifically excluded from the term “isomers”, as used herein, are structural (or constitutional) isomers (i.e. isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g., C₁₋₇ alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, N-nitroso/hyroxyazo, and nitro/aci-nitro.

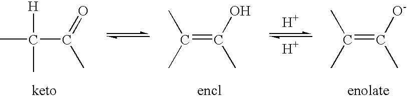

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic ¹⁶O and ¹⁸O; and the like.

Unless otherwise specified, a reference to a particular compound includes all such isomeric forms, including (wholly or partially) racemic and other mixtures thereof. Methods for the preparation (e.g. asymmetric synthesis) and separation (e.g., fractional crystallisation and chromatographic means) of such isomeric forms are either known in the art or are readily obtained by adapting the methods taught herein, or known methods, in a known manner.

The compound of the first aspect of the invention includes isomers of formula (I) as described above. For example, 2-((2S,6S)-2,6-Dimethyl-morpholin-4-yl)-N-[5-(6-morpholin-4-yl-4-oxo-4H-pyran-2-yl)-9H-thioxanthen-2-yl]-acetamide and 2-((2R,6R)-2,6-Dimethyl-morpholin-4-yl)-N-[5-(6-morpholin-4-yl-4-oxo-4H-pyran-2-yl)-9H-thioxanthen-2-yl]-acetamide. Particularly preferred is the isomeric form corresponding to 2-((2R,6S)-2,6-Dimethyl-morpholin-4-yl)-N-[5-(6-morpholin-4-yl-4-oxo-4H-pyran-2-yl)-9H-thioxanthen-2-yl]-acetamide.

Unless otherwise specified, a reference to a particular compound also includes ionic, salt, solvate, and protected forms of thereof, for example, as discussed below.

It may be convenient or desirable to prepare, purify, and/or handle a corresponding salt of the active compound, for example, a pharmaceutically-acceptable salt. Examples of pharmaceutically acceptable salts are discussed in Berge, et al., J. Pharm. Sci., 66, 1-19 (1977).

For example, a functional group of the compound which may be anionic can be used to form a salt with a suitable cation. Examples of suitable inorganic cations include, but are not limited to, alkali metal ions such as Na⁺and K⁺, alkaline earth cations such as Ca²⁺and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Similarly, a functional group of the compound which may be cationic may be used to form a salt with a suitable anion. Examples of suitable inorganic anions include, but are not limited to, those derived from the following inorganic acids: hydrochloric, hydrobromic, hydroiodic, sulphuric, sulphurous, nitric, nitrous, phosphoric, and phosphorous. Examples of suitable organic anions include, but are not limited to, those derived from the following organic acids: acetic, propionic, succinic, glycolic, stearic, palmitic, lactic, malic, pamoic, tartaric, citric, gluconic, ascorbic, maleic, hydroxymaleic, phenylacetic, glutamic, aspartic, benzoic, cinnamic, pyruvic, salicyclic, sulfanilic, 2-acetyoxybenzoic, fumaric, phenylsulfonic, toluenesulfonic, methanesulfonic, ethanesulfonic, ethane disulfonic, oxalic, pantothenic, isethionic, valeric, lactobionic, and gluconic. Examples of suitable polymeric anions include, but are not limited to, those derived from the following polymeric acids: tannic acid, carboxymethyl cellulose.

It may be convenient or desirable to prepare, purify, and/or handle a corresponding solvate of the active compound. The term “solvate” is used herein in the conventional sense to refer to a complex of solute (e.g. active compound, salt of active compound) and solvent. If the solvent is water, the solvate may be conveniently referred to as a hydrate, for example, a mono-hydrate, a di-hydrate, a tri-hydrate, etc.

It may be convenient or desirable to prepare, purify, and/or handle the active compound in a chemically protected form. The term “chemically protected form”, as used herein, pertains to a compound in which one or more reactive functional groups are protected from undesirable chemical reactions, that is, are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, Protective Groups in Organic Synthesis (T. Green and P. Wuts, Wiley, 1999).

For example, an aldehyde or ketone group may be protected as an acetal or ketal, respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid.

It may be convenient or desirable to prepare, purify, and/or handle the active compound in the form of a prodrug. The term “prodrug”, as used herein, pertains to a compound which, when metabolised (e.g. in vivo), yields the desired active compound. Typically, the prodrug is inactive, or less active than the active compound, but may provide advantageous handling, administration, or metabolic properties.

For example, some prodrugs are esters of the active compound (e.g. a physiologically acceptable metabolically labile ester). During metabolism, the ester group is cleaved to yield the active drug.

Also, some prodrugs are activated enzymatically to yield the active compound, or a compound which, upon further chemical reaction, yields the active compound. For example, the prodrug may be a sugar derivative or other glycoside conjugate, or may be an amino acid ester derivative.

Acronyms

For convenience, many chemical moieties are represented using well known abbreviations, including but not limited to, methyl (Me), ethyl (Et), n-propyl (nPr), iso-propyl (iPr), n-butyl (nBu), tert-butyl (tBu), n-hexyl (nHex), cyclohexyl (cHex), phenyl (Ph), biphenyl (biPh), benzyl (Bn), naphthyl (naph), methoxy (MeO), ethoxy (EtO), benzoyl (Bz), acetyl (Ac), 1,3-bis(diphenylphosphino) propane (dppf).

For convenience, many chemical compounds are represented using well known abbreviations, including but not limited to, methanol (MeOH), ethanol (EtOH), iso-propanol (i-PrOH), methyl ethyl ketone (MEK), ether or diethyl ether (Et₂O), acetic acid (AcOH), dichloromethane (methylene chloride, DCM), trifluoroacetic acid (TFA), dimethylformamide (DMF), tetrahydrofuran (THF), and dimethylsulfoxide (DMSO).

CHK and ATM Combination

The term “combination” refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the benefit of the synergistic and/or additive effect of the combination.

Treatment

Where cancer is referred to, it can refer to the treatment of oesophageal cancer, myeloma, hepatocellular, pancreatic, cervical cancer, ewings tumour, neuroblastoma, kaposis sarcoma, ovarian cancer, breast cancer, colorectal cancer, prostate cancer, bladder cancer, melanoma, lung cancer-non small cell lung cancer (NSCLC), and small cell lung cancer (SCLC), gastric cancer, head and neck cancer, brain cancer such as glioblastoma, renal cancer, lymphoma, stomach, cervix, colon, thyroid and skin and leukaemia. The combination can also be used to treat hematopoietic tumors of lymphoid lineage, including acute lymphocytic leukaemia, B-cell lymphoma and Burketts lymphoma, hematopoietic tumours of myeloid lineage, including acute and chronic myelogenous leukaemias and promyelocytic leukaemia; tumours of mesenchymal origin, including fibrosarcoma and rhabdomyosarcoma; and other tumours, including melanoma, seminoma, tetratocarcinoma, neuroblastoma and glioma.

The treatment of cancer also refers to treatment of an established primary tumour or tumours and developing primary tumour or tumours. In another aspect of the invention the treatment of cancer relates to the treatment of metastases. In another aspect of the invention the treatment of cancer relates to treatment of an established primary tumour or tumours or developing primary tumour or tumours.

Therefore according to the present invention, there is provided a method of treating cancer, in a warm-blooded animal, such as man, in need of such treatment which comprises administering to said animal an effective amount of a CHK inhibitor, or a pharmaceutically acceptable salt thereof in combination with an effective amount of an ATM inhibitor, or a pharmaceutically acceptable salt thereof.

According to an aspect of the invention there is provided a pharmaceutical composition that include a CHK inhibitor, or a pharmaceutically acceptable salt thereof, in combination with a pharmaceutical composition which comprises an ATM inhibitor, or a pharmaceutically acceptable salt thereof for use in the treatment of cancer.

According to another feature of the invention there is provided the use of a CHK inhibitor, or a pharmaceutically acceptable salt thereof, in combination with an ATM inhibitor, or a pharmaceutically acceptable salt thereof, in the manufacture of a medicament for use in the treatment of cancer, in a warm-blooded animal, such as man.

According to yet another feature of the invention there is provided the use of a CHK inhibitor, or a pharmaceutically acceptable salt thereof, in combination with an ATM inhibitor, or a pharmaceutically acceptable salt thereof, for use in the treatment of cancer, in a warm-blooded animal, such as man.

Another aspect of the invention provides for the use of a CHK inhibitor, or a pharmaceutically acceptable salt thereof, in combination with a ATM inhibitor, or a pharmaceutically acceptable salt thereof, in the preparation of a medicament for use as an adjunct in cancer therapy or for potentiating tumour cells for treatment with ionizing radiation or chemotherapeutic agents.

Other further aspects of the invention provide inhibiting ATM and/or CHK activity, comprising administering to a subject a CHK inhibitor, or a pharmaceutically acceptable salt thereof, in combination with a ATM inhibitor, or a pharmaceutically acceptable salt thereof.

Other further aspects of the invention provide inhibiting cell proliferation, comprising administering to a subject a CHK inhibitor, or a pharmaceutically acceptable salt thereof, in combination with a ATM inhibitor, or a pharmaceutically acceptable salt thereof.

The pharmaceutical compositions may be in a form suitable for oral administration, for example as a tablet or capsule, for parenteral injection (including intravenous, subcutaneous, intramuscular, intravascular or infusion), as a sterile solution, suspension or emulsion, for topical administration as an ointment or cream or for rectal administration as a suppository.

Preferably the combination is administered separately, one after another. In one embodiment, the ATM inhibitor is administered orally and the CHK inhibitor is administered intravenously. In another embodiment, CHK and ATM are both administered orally.

The CHK inhibitor, or a pharmaceutically acceptable salt thereof, will normally be administered to a warm-blooded animal at a unit dose of 1 g or less daily but more than 2.5 mg and this would be expected to provide a therapeutically-effective dose. However the daily dose will necessarily be varied depending upon the host treated, the particular route of administration, and the severity of the illness being treated. Accordingly the optimum dosage may be determined by the practitioner who is treating any particular patient. Particularly the CHK could be administered to a warm-blooded animal, at a unit dose of less than 250 mg per day. In another aspect of the invention, the CHK could be administered to a warm-blooded animal, at a unit dose of less than 130 mg per day. In a further aspect of the invention, the CHK could be administered to a warm-blooded animal, at a unit dose of less than 50 mg per day.

The ATM inhibitor, or pharmaceutically acceptable salt thereof, will normally be administered to a warm-blooded animal at a unit dose, for example, from about 20 mg to 1 g of active ingredient. The ATM can be formulated in a conventional tablet for oral administration containing 50 mg, 100 mg, 250 mg or 500 mg of active ingredient. Conveniently the daily oral dose is above 150 mg, for example, in the range 150 to 750 mg, preferably in the range 200 to 500 mg. For a single dosage form, the active ingredients may be compounded with an appropriate and convenient amount of excipients which may vary from about 5 to about 98 percent by weight of the total composition. Dosage unit forms will generally contain about 20 mg to about 500 mg of each active ingredient. However the daily dose will necessarily be varied depending upon the host treated, the particular route of administration, and the severity of the illness being treated. Accordingly the optimum dosage may be determined by the practitioner who is treating any particular patient.

According to a further aspect of the present invention there is provided a kit comprising a CHK inhibitor, as described above, or a pharmaceutically acceptable salt thereof, and an ATM inhibitor as described above, or a pharmaceutically acceptable salt thereof; optionally with instructions for use; for use in the treatment of cancer.

### EXAMPLES

### CHK-ATM Combination

Combination experiments were carried out to assess the ability of a CHK inhibitor, 5-(3-Fluoro-phenyl)-3-ureido-thiophene-2-carboxylic acid (S)-piperidin-3-ylamide, to sensitize cells to an ATM inhibitor, 2-[(2,6)-2,6-dimethylmorpholin-4-yl]-N-[5-(6-morpholin-4-yl-4-oxo-4H-pyran-2-yl)-9H-thioxanthen-2-yl]acetamide, using a cell viability endpoint.

Cell lines chosen are relatively insensitive to either compound when used as a single agent. Specifically, two cell lines with inactive p53, which have previously been determined to be more sensitive to CHK inhibition than cells expressing wild type p53, were used. The effect of simultaneous compound addition and exposure versus sequential addition of compounds followed by simultaneous exposure was examined.

The cell lines used in this study were SW620, which endogenously express mutant p53, and NCI-H460dnp53, which are stably transfected to express dominant negative p53. Cells were seeded in 96-well plates on day 0 and treated with either a single drug or simultaneously with both drugs for 4 days beginning on Day 1. For sequential addition, CHK inhibitor was added to cells for 24 hours beginning on Day 1, followed by addition of ATM inhibitor on Day 2, and exposure to both drugs continued for an additional 72 hours. The CHK inhibitor was used at a constant concentration that had been previously determined to have no activity when used as a single agent. Cells were dosed with the ATM inhibitor to generate a full dose-response curve. See Table 1 for an example of the drug concentrations used in each cell line. In these experiments, CHK inhibitor was added manually and ATM inhibitor was added using automation.

TABLE 1
Compound Concentrations Used (uM)

| | | Top concentration |
|---|---|---|
| Cell Line | CHK inhibitor | used for ATM inihitor |
| SW620 | 0.28 | 30 |
| NCI-H460dnp53 | 0.33 | 30 |

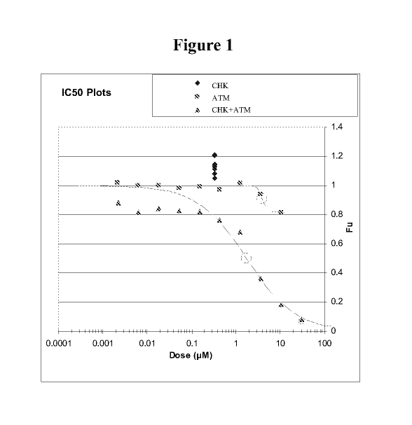

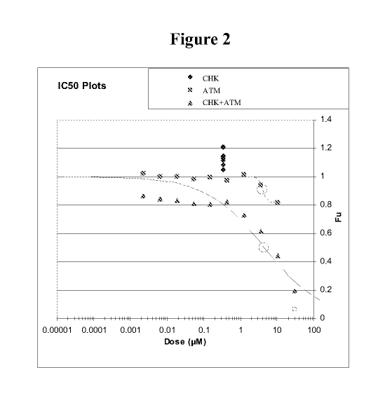

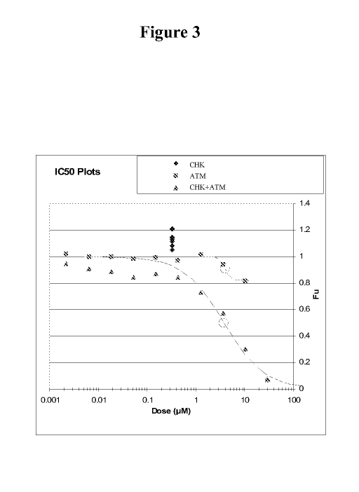

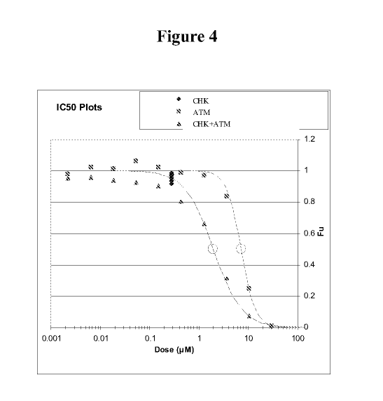

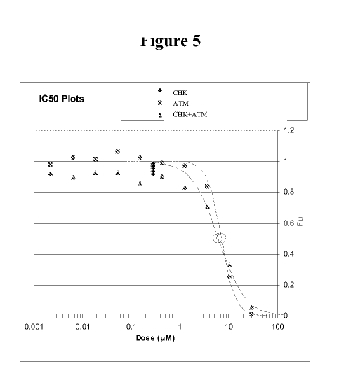

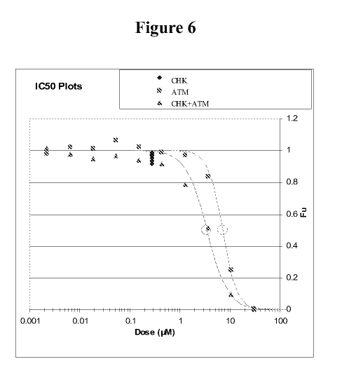

Cell viability was measured on day 5 using an MTS colorimetric assay. Dose (concentration of drug used, uM) versus fraction unaffected (Fu) for ATM inhibitor alone versus in combination were plotted. Replicate points for the constant concentration of CHK were also plotted. Data were analyzed by comparing the dose-response curve of the single agent to that for the combination (see Table 2 and FIGS. 1-6).

TABLE 2
Representative IC50s* of ATM inhibitor (uM) used as a single agent
vs. in combination with AZD7762

| Cell Line or | | ATM inhibitor + | CHK inhibitor | ATM inhibitor |
|---|---|---|---|---|
| Number of | ATM inhibitor | CHK inhibitor | followed by | followed by CHK |
| Experiments | alone | Simultaneous | ATM inhibitor | inhibitor |
| NCI-H460dnp53 | >30 uM | 1.7 | 4.4 | 3.8 |
| SW620 | 7.0 | 1.9 | 6.0 | 3.4 |
| Number of | 2 | 2 | 2 | 2 |
| experiments | | | | |

*IC = Inhibitory Concentration

1. A combination comprising a checkpoint kinase (CHK) inhibitor, or a pharmaceutically acceptable salt thereof, and an ataxia telangiectasia mutated (ATM) inhibitor, or a pharmaceutically acceptable salt thereof.

2. A combination according to claim 1 wherein the checkpoint kinase (CHK) inhibitor is selected from a compound of formula (I): 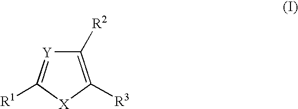
wherein:
X is selected from NH, S and O;
Y is selected from CH or N;
R¹ is selected from cyano, isocyano, C₁₋₆alkyl, —NK¹¹R¹², C₁₋₆alkoxy, C₂₋₆alkenyl, C₂₋₆alkynyl, cycloalkyl, cycloalkenyl, aryl, and heterocyclyl, provided R¹ is not thienyl; and wherein R¹ may be optionally substituted on one or more carbon atoms by one or more R⁹; and wherein if said R¹ contains an —NH— moiety, the nitrogen of said moiety may be optionally substituted by a group selected from R¹⁰;
R² and R³ are each independently selected from —C(═O)NR⁶R⁷, —SO₂NR¹⁶R¹⁷, —NHC(═O)NHR⁴, and —NHC(═NR⁸)NH₂;
R⁴ is selected from H, OH, —NR¹¹R¹², benzyl, C₁₋₆alkoxy, cycloalkyl, cylcoalkenyl, aryl, heterocyclyl, mercapto, CHO, —COaryl, —CO(C₁₋₆alkyl), —CONR³⁰R³¹, —CO₂(C₁₋₆alkyl), —CO₂aryl, —CO₂NR³⁰R³¹, —Salkyl, —SO(C₁₋₆alkyl), —SO₂(C₁₋₆alkyl), —Saryl, —SOaryl, —SO₂aryl, —SO₂NR³⁰R³¹, and —(C₁₋₆alkyl)SO₂ NR³⁰R³¹ wherein R⁴ may be optionally substituted on one or more carbon atoms by one or more R¹⁵; and wherein if said heterocyclyl contains a —NH— moiety, the nitrogen may be optionally substituted by a group selected from R¹⁴;
R⁶ and R⁷ are each independently selected from H, OH, OCH₃, C₁₋₆alkoxy, —NH₂, —NHCH₃, —N(CH₃)₂, (C₁₋₃alkyl)NR¹¹R¹², —CH₂CH₂OH, cycloalkyl, and a 5, 6, or 7-membered heterocyclyl ring containing at least one nitrogen atom, provided R⁶ and R⁷ are not both H; alternatively R⁶ and R⁷ taken together with the N to which they are attached form a heterocyclic ring; wherein R⁶ and R⁷ independently of each other may be optionally substituted on one or more carbon atoms by one or more R¹⁸; and wherein if said heterocyclyl contains a —NH— moiety, the nitrogen of said moiety may be optionally substituted by a group selected from R¹⁹;
R⁸ is selected from cyano, isocyano, —SO₂(C₁₋₆alkyl), —SO₂-aryl; —SO₂cycloalkyl, —SO₂cycloalkenyl, —SO₂heterocyclyl, and CF₃; wherein R⁸ may be optionally substituted on one or more carbon atoms by one or more R²³;
R⁹, R¹⁵, R¹⁸, R²³, R²⁴ and R³³ are each independently selected from halogen, nitro, —NR³⁰R³¹, cyano, isocyano, C₁₋₆alkyl, C₂₋₆alkenyl, C₂₋₆alkynyl, aryl, cycloalkyl, heterocyclyl, hydroxy, keto(═O), —O(C₁₋₆alkyl), —Oaryl, —OCOalkyl, —NHCHO, —N(C₁₋₆alkyl)CHO, —NHCONR³⁰R³¹, —N(C₁₋₆alkyl)CONR³⁰R³¹, —NHCOalkyl, —NHCO₂(C₁₋₆alkyl); —NHCO₂H, —N(C₁₋₆alkyl)CO(C₁₋₆alkyl), —NHSO₂(C₁₋₆alkyl), carboxy, -amidino, —CHO, —CONR³⁰R³¹, —CO(C₁₋₆alkyl), —COheterocyclyl, —COcycloalkyl, —CO₂H, —CO₂(C₁₋₆alkyl), —CO₂(aryl), —CO₂(NR³⁰R³¹), mercapto, —S(C₁₋₆alkyl), —SO(C₁₋₆alkyl), —SO₂(C₁₋₆alkyl), —SO₂NR³⁰R³¹; wherein R⁹, R^{‥}, R¹⁸, R²³, R²⁴ and R³³ independently of each other may be optionally substituted on carbon by one or more R²⁰ and on nitrogen of any moiety that contains an NH or NH₂ by R²¹;
R¹⁰, R¹⁴, R¹⁹, R²⁵ and R³⁴ are each independently selected from halogen, nitro, —NR³⁰R³¹, cyano, isocyano, C₁₋₆alkyl, C₂₋₆alkenyl, C₂₋₆alkynyl, aryl, cycloalkyl, heterocyclyl, hydroxy, keto(═O), —O(C₁₋₆alkyl), —Oaryl, —OCOalkyl, —NHCHO, —N(C₁₋₆alkyl)CHO, —NHCONR³⁰R³¹, —N(C₁₋₆alkyl)CONR³⁰R³¹, —NHCOalkyl, —NHCO₂(C₁₋₆alkyl); —NHCO₂H, —N(C₁₋₆alkyl)CO(C₁₋₆alkyl), —NHSO₂(C₁₋₆alkyl), carboxy, -amidino, —CHO, —CONR³⁰R³¹, —CO(C₁₋₆alkyl), —COheterocyclyl, —COcycloalkyl, —CO₂H, —CO₂(C₁₋₆alkyl), —CO₂(aryl), —CO₂(NR³⁰R³¹), mercapto, —S(C₁₋₆alkyl), —SO(C₁₋₆alkyl), —SO₂(C₁₋₆alkyl), —SO₂NR³⁰R³¹; wherein R¹⁰, R¹⁴, R¹⁹, R²⁵ and R³⁴ independently of each other may be optionally substituted on carbon by one or more R²² and on nitrogen of any moiety that contains an NH or NH₂ by R²³;
R¹¹ and R¹² are independently selected from H, C₁₋₆alkyl, cycloalkyl, aryl, heterocyclyl; alternatively R¹¹ and R¹² taken together with the N to which they are attached form a heterocyclic ring; wherein and R¹² independently of each other may be optionally substituted on carbon by one or more R³³; and wherein if said heterocyclyl contains a —NH— moiety, the nitrogen of said moiety may be optionally substituted by a group selected from R³⁴;
R¹⁶ and R¹⁷ are each independently selected from H, OH, OCH₃, C₁₋₆alkoxy, NH₂, —NHCH₃, —N(CH₃)₂, (C₁₋₃alkyl)NR¹¹R¹², —CH₂CH₂OH, cycloalkyl, aryl, or a 5, 6 or 7-membered heterocyclyl ring containing at least one nitrogen atom, provided R¹⁶ and R¹⁷ are not both H; alternatively R¹⁶ and R¹⁷ taken together with the N to which they are attached form an optionally substituted heterocyclic ring; wherein R¹⁶ and R¹⁷ independently of each other may be optionally substituted on one or more carbon atoms by one or more R²⁴; and wherein if said heterocyclyl contains an —NH— moiety, the nitrogen of said moiety may be optionally substituted by a group selected from R²⁵;
R²⁰, R²² and R³² are each independently selected from halogen, nitro, —NR³⁰R³¹, cyano, isocyano, C₁₋₆alkyl, C₂₋₆alkenyl, C₂₋₆alkynyl, aryl, cycloalkyl, heterocyclyl, hydroxy, keto(═O), —O(C₁₋₆alkyl), —Oaryl, —OCOalkyl, —NHCHO, —N(C₁₋₆alkyl)CHO, —NHCONR³⁰R³¹, —N(C₁₋₆alkyl)CONR³⁰R³¹, —NHCOalkyl, —NHCO₂(C₁₋₆alkyl); —NHCO₂H, —N(C₁₋₆alkyl)CO(C₁₋₆alkyl), —NHSO₂(C₁₋₆alkyl), carboxy, -amidino, —CHO, —CONR³⁰R³¹, —CO(C₁₋₆alkyl), —COheterocyclyl, —COcycloalkyl, —CO₂H, CO₂(C₁₋₆alkyl), CO₂(aryl), —CO₂(NR³⁰R³¹), mercapto, —S(C₁₋₆alkyl), —SO(C₁₋₆alkyl), —SO₂(C₁₋₆alkyl), —SO₂NR³⁰R³¹; wherein R²⁰, R²¹ and R³² independently of each other may be optionally substituted on carbon by one or more R²⁶ and on nitrogen of any moiety that contains an NH or NH₂ by R²⁷;
R²¹, R²³ and R³⁵ are each independently selected from halogen, nitro, —NR³⁰R³¹, cyano, isocyano, C₁₋₆alkyl, C₂₋₆alkenyl, C₂₋₆alkynyl, aryl, cycloalkyl, heterocyclyl, hydroxy, keto(═O), —O(C₁₋₆alkyl), —Oaryl, —OCOalkyl, —NHCHO, —N(C₁₋₆alkyl)CHO, —NHCONR³⁰R³¹, —N(C₁₋₆alkyl)CONR³⁰R³¹, —NHCOalkyl, —NHCO₂(C₁₋₆alkyl); —NHCO₂H, —N(C₁₋₆alkyl)CO(C₁₋₆alkyl), —NHSO₂(C₁₋₆alkyl), carboxy, -amidino, —CHO, —CONR³⁰R³¹, —CO(C₁₋₆alkyl), —COheterocyclyl, —COcycloalkyl, —CO₂H, —CO₂(C₁₋₆alkyl), —CO₂(aryl), —CO₂(NR³⁰R³¹), mercapto, —S(C₁₋₆alkyl), —SO(C₁₋₆alkyl), —SO₂(C₁₋₆alkyl), —SO₂NR³⁰R³¹; wherein R²¹, R²³ and R³⁵ independently of each other may be optionally substituted on carbon by one or more R²⁸ and on nitrogen of any moiety that contains an NH by R²⁹;
R²⁶ and R²⁸ are each independently selected from halogen, nitro, —NR³⁰R³¹, cyano, isocyano, C₁₋₆alkyl, C₂₋₆alkenyl, C₂₋₆alkynyl, aryl, cycloalkyl, heterocyclyl, hydroxy, keto(═O), —O(C₁₋₆alkyl), —Oaryl, —OCOalkyl, —NHCHO, —N(C₁₋₆alkyl)CHO, —NHCONR³⁰R³¹, —N(C₁₋₆alkyl)CONR³⁰R³¹, —NHCOalkyl, —NHCO₂(C₁₋₆alkyl); —NHCO₂H, —N(C₁₋₆alkyl)CO(C₁₋₆alkyl), —NHSO₂(C₁₋₆alkyl), carboxy, -amidino, —CHO, —CONR³⁰R³¹, —CO(C₁₋₆alkyl), —COheterocyclyl, —COcycloalkyl, —CO₂H, —CO₂(C₁₋₆alkyl), —CO₂(aryl), —CO₂(NR³⁰R³¹), mercapto, —S(C₁₋₆alkyl), —SO(C₁₋₆alkyl), —SO₂(C₁₋₆alkyl), —SO₂NR³⁰R³¹;
R²⁷ and R²⁹ are each independently selected from halogen, nitro, —NR³⁰R³¹, cyano, isocyano, C₁₋₆alkyl, C₂₋₆alkenyl, C₂₋₆alkynyl, aryl, cycloalkyl, heterocyclyl, hydroxy, keto(═O),
—O(C₁₋₆alkyl), —Oaryl, —OCOalkyl, —NHCHO, —N(C₁₋₆alkyl)CHO, —NHCONR³⁰R³¹, —N(C₁₋₆alkyl)CONR³⁰R³¹, —NHCOalkyl, —NHCO₂(C₁₋₆alkyl); —NHCO₂H, —N(C₁₋₆alkyl)CO(C₁₋₆alkyl), —NHSO₂(C₁₋₆alkyl), carboxy, -amidino, —CHO, —CONR³⁰R³¹, —CO(C₁₋₆alkyl), —COheterocyclyl, —COcycloalkyl, —CO₂H, —CO₂(C₁₋₆alkyl), —CO₂(aryl), —CO₂(NR³⁰R³¹), mercapto, —S(C₁₋₆alkyl), —SO(C₁₋₆alkyl), —SO₂(C₁₋₆alkyl), —SO₂NR³⁰R³¹;
R³⁰ and R³¹ are each independently selected from halogen, nitro, —NH₂, cyano, isocyano, C₁₋₆alkyl, C₂₋₆alkenyl, C₂₋₆alkynyl, aryl, cycloalkyl, heterocyclyl, hydroxy, keto(═O),
—O(Cl₁₋₆alkyl), —Oaryl, —OCOalkyl, —NHCHO, —N(C₁₋₆alkyl)CHO, —NHCONR¹¹R¹², —N(C₁₋₆alkyl)CONR¹¹R¹², —NHCOalkyl, —NHCO₂(C₁₋₆alkyl); —NHCO₂H, —N(C₁₋₆alkyl)CO(C₁₋₆alkyl), —NHSO₂(C₁₋₆alkyl), carboxy, -amidino, —CHO, —CONR³⁰R³¹, —CO(C₁₋₆alkyl), —COheterocyclyl, —COcycloalkyl, —CO₂H, —CO₂(C₁₋₆alkyl), —CO₂(aryl), —CO₂(NR³⁰R³¹), mercapto, —S(C₁₋₆alkyl), —SO(C₁₋₆alkyl), —SO₂(C₁₋₆alkyl), —SO₂NR¹¹R¹²; wherein R³⁰ and R³¹ independently of each other may be optionally substituted on carbon by one or more R³²; and wherein if said heterocyclyl contains a —NH— or NH₂ moiety, the nitrogen of said moiety may be optionally substituted by a group selected from R³⁵;
or a pharmaceutically acceptable salt thereof;
provided that when X is S; Y is CH; R₂ is C(═O)NR⁶R⁷; and R³ is NHC(═O)NHR⁴; then R¹ cannot be 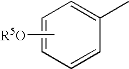
wherein R⁵ is selected from H, optionally substituted carbocyclyl, or optionally substituted C₁₋₆alkyl; with the further proviso that said compound is not
5-Methyl-2-ureido-thiophene-3-carboxylic acid (1-ethyl-piperidin-3-yl)-amide;
[3-((S)-3-Amino-azepane-1-carbonyl)-5-ethyl-thiophen-2-yl]-urea;
2-Morpholin-4-yl-4-ureido-thiazole-5-carboxylic acid (S)-piperidin-3-ylamide;
2-Methyl-5-ureido-oxazole-4-carboxylic acid (S)-piperidin-3-ylamide;
5-(4-Chloro-phenyl)-3-{3-[(R)-1-(2,2,2-trifluoro-acetyl)-piperidin-3-yl]-ureido}-thiophene-2-carboxylic acid (S)-piperidin-3-ylamide; or
N-(3-{[(3S)-3-aminoazepan-1-yl]carbonyl}-5-pyridin-2-yl-2-thienyl)urea; or a pharmaceutically acceptable salt thereof.

3. A combination according to claim 1 wherein the ATM is selected from a compound of formula (Ib): 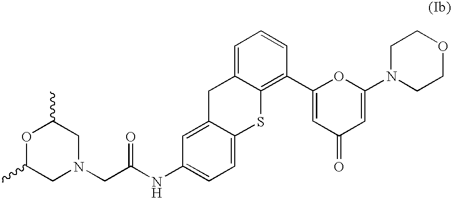
or a pharmaceutically acceptable salt thereof.

4. A pharmaceutical composition comprising a combination according to claims 1-3, in association with a pharmaceutically acceptable diluent or carrier.

5. A method of treating cancer in a warm-blooded animal in need of such treatment which comprises administering to said animal an effective amount of a combination according to any one of claims 1-3.

6.-8. (canceled)

9. The method according to claim 5, wherein the cancer is oesophageal cancer, myeloma, hepatocellular, pancreatic, cervical cancer, ewings tumour, neuroblastoma, kaposis sarcoma, ovarian cancer, breast cancer, colorectal cancer, prostate cancer, bladder cancer, melanoma, lung cancer—non small cell lung cancer (NSCLC), and small cell lung cancer (SCLC), gastric cancer, head and neck cancer, brain cancer, renal cancer, lymphoma and leukaemia.

10. The method or use or combination according to claim 9 wherein the cancer is in a metastatic state.

11. The method or use or combination according to claim 9 wherein the cancer is in a non-metastatic state.

12. The method or use or combination according to claim 9 wherein the cancer is renal, thyroid, lung, breast or prostate cancer that is producing bone metastases.
